(19) United States
(12) Patent Application Publication
(10) Pub. No.: US 2026/0069236 A1
(43) Pub. Date: Mar. 12, 2026

(54) RADIOGRAPHIC IMAGING APPARATUS, RADIOGRAPHIC IMAGING METHOD, AND STORAGE MEDIUM

(71) Applicant: KONICA MINOLTA, INC., Tokyo (JP)

(72) Inventor: Tatsuya TAKAGI, Tokyo (JP)

(73) Assignee: KONICA MINOLTA, INC., Tokyo (JP)

(21) Appl. No.: 19/308,795

(22) Filed: Aug. 25, 2025

(30) Foreign Application Priority Data
Sep. 9, 2024 (JP) 2024-154521

(51) Int. Cl. A61B 6/58; A61B 6/00; A61B 6/40; G16H 40/63

(52) U.S. Cl. CPC A61B 6/589; A61B 6/40; A61B 6/547; G16H 40/63

(57) ABSTRACT

A radiographic imaging apparatus includes: an optical camera that obtains optical images; a first change section that changes a relative position of a tube with respect to a subject; and a hardware processor. The hardware processor determines whether relative positioning of the tube with respect to the subject is good, based on the optical images, and determines the relative position of the tube with respect to the subject, based on the determination on the positioning.

The entire disclosure of Japanese Patent Application No.2024-154521, filed on September 9, 2024, is incorporated herein by reference in its entirety.

### TECHNICAL FIELD

The present disclosure relates to a radiographic imaging apparatus, a radiographic imaging method, and a storage medium.

### BACKGROUND OF THE INVENTION

Conventionally, a user such as a radiographer manually positions a tube and a subject to perform radiographic imaging. In recent years, development of a radiographic imaging apparatus that automatically positions a tube with respect to a subject has been started.

Japanese Unexamined Patent Publication No. 2012-24399 describes imaging a subject by using an optical camera integrally formed with a bulb and determining whether the relative positional relation between the bulb and the subject is correct.

Further, Japanese Unexamined Patent Publication No. 2023-102361 describes imaging a subject once by using an optical camera and obtaining positional information between the tube and the subject.

According to JP2012-24399A, if it is determined that the relative positional relation between the tube and the subject is incorrect, the radiologist repositions the subject.

In a case where the radiographic imaging apparatus automatically repositions the bulb, the apparatus needs to obtain not only the correctness/incorrectness information of the relative positional relation but also relative positional information between the bulb and the subject as a deviation amount.

JP2023-102361A performs imaging only once by using the optical camera and cannot obtain correct positional information between the tube and the subject as a deviation amount.

An object of the present disclosure is to automatically optimize determining the relative positions between the tube and the subject.

### SUMMARY OF THE INVENTION

To achieve at least one of the abovementioned objects, according to an aspect of the present disclosure, there is provided a radiographic imaging apparatus including: an optical camera that obtains optical images; a first change section that changes a relative position of a tube with respect to a subject; and a hardware processor, wherein the hardware processor determines whether relative positioning of the tube with respect to the subject is good, based on the optical images, and determines the relative position of the tube with respect to the subject, based on the determination on the positioning.

According to another aspect of the present disclosure, there is provided a radiographic imaging method for a radiographic imaging apparatus that includes an optical camera that obtains optical images and a first change section that changes a relative position of a tube with respect to a subject, the method including: determining whether relative positioning of the tube with respect to the subject is good, based on the optical images; and determining the relative position of the tube with respect to the subject, based on the determination on the positioning.

According to another aspect of the present disclosure, there is provided a non-transitory computer-readable storage medium storing a program for a radiographic imaging apparatus that includes an optical camera that obtains optical images and a first change section that changes a relative position of the tube with respect to a subject, the program causing a computer of the radiographic imaging apparatus to: determine whether relative positioning of the tube with respect to the subject is good, based on the optical images and determine the relative position of the tube with respect to the subject, based on the determination on the positioning

### BRIEF DESCRIPTION OF DRAWINGS

The advantages and features provided by one or more embodiments of the invention will become more fully understood from the detailed description given hereinafter and the appended drawings which are given by way of illustration only, and thus are not intended as a definition of the limits of the present disclosure, and wherein:

FIG. 1 illustrates a schematic configuration of a radiographic imaging apparatus of the present embodiment;

FIG. 2 illustrates a schematic configuration of the radiographic imaging apparatus of the present embodiment;

FIG. 3 illustrates a schematic configuration of the radiographic imaging apparatus of the present embodiment;

FIG. 4 illustrates a schematic configuration of the radiographic imaging apparatus of the present embodiment;

FIG. 5 illustrates a block diagram of a configuration of the radiographic imaging apparatus;

FIG. 6 is a flowchart of a positioning determination process; and

FIG. 7 is a flowchart of the positioning determining process.

### DETAILED DESCRIPTION

Hereinafter, an embodiment of the present disclosure is described with reference to the drawings. However, the embodiment described below has various limitations which are technically preferable for carrying out the present disclosure. Therefore, the technical scope of the present disclosure is not limited to the following embodiment and illustrated examples.

### <Overall Configuration of Radiographic Imaging Apparatus 1>

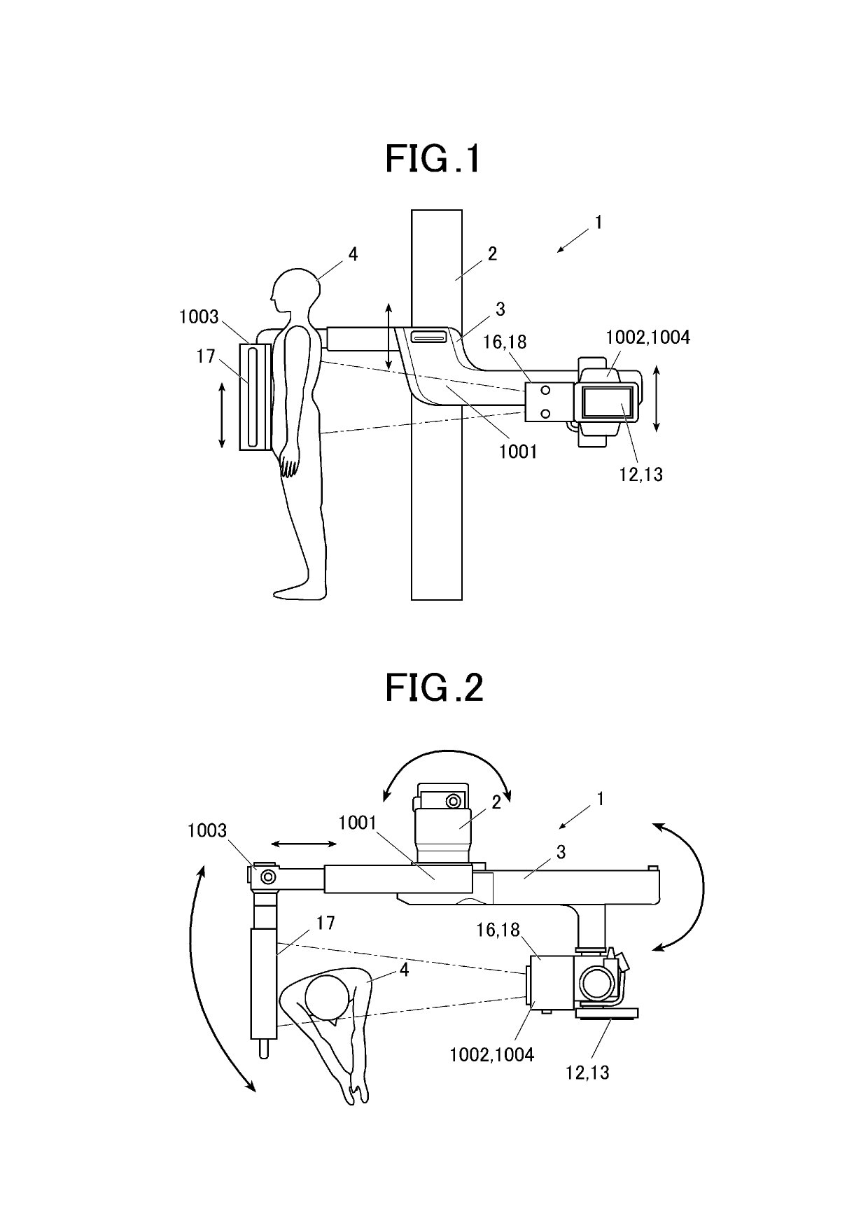

First, a schematic configuration of a radiographic imaging apparatus 1 according to the present embodiment will be described with reference to FIG. 1 and FIG. 2. FIG. 1 is a schematic configuration diagram of the radiographic imaging apparatus 1 when viewed from the side. FIG. 2 is a schematic configuration diagram of the radiographic imaging apparatus 1 when viewed from above.

The radiographic imaging apparatus 1 includes a support column 2 and a structure 3 movably fixed to the support column 2.

The structure 3 includes a first movement mechanism 1001 that moves the structure 3 in the up-down direction as illustrated in FIG. 1 or in the rotation direction around the support column 2 as illustrated in FIG. 2.

The structure 3 includes a tube 16, a flat panel detector (FPD) 17, and an optical camera 18, which will be described later.

The tube 16, the FPD 17, and the optical camera 18 are each movable up and down, back and forth, right and left, and in the rotation direction (a direction of inclination with respect to the subject 4) with respect to the subject 4.

The tube 16 includes a second movement mechanism 1002 that moves the tube 16 up and down, back and forth, right and left, and in the rotation direction.

The FPD 17 includes a third movement mechanism 1003 that moves the FPD 17 up and down, back and forth, left and right, and in the rotation direction. The third movement mechanism 1003 moves the FPD 17 to a position where the FPD 17 faces the tube 16 according to the movement of the tube 16.

The optical camera 18 includes a fourth movement mechanism 1004 that moves the optical camera 18 up and down, back and forth, left and right, and in the rotation direction.

In the radiographic imaging apparatus 1 illustrated in FIGS. 1 and 2, the tube 16 and the optical camera 18 are integral and movable as a unit.

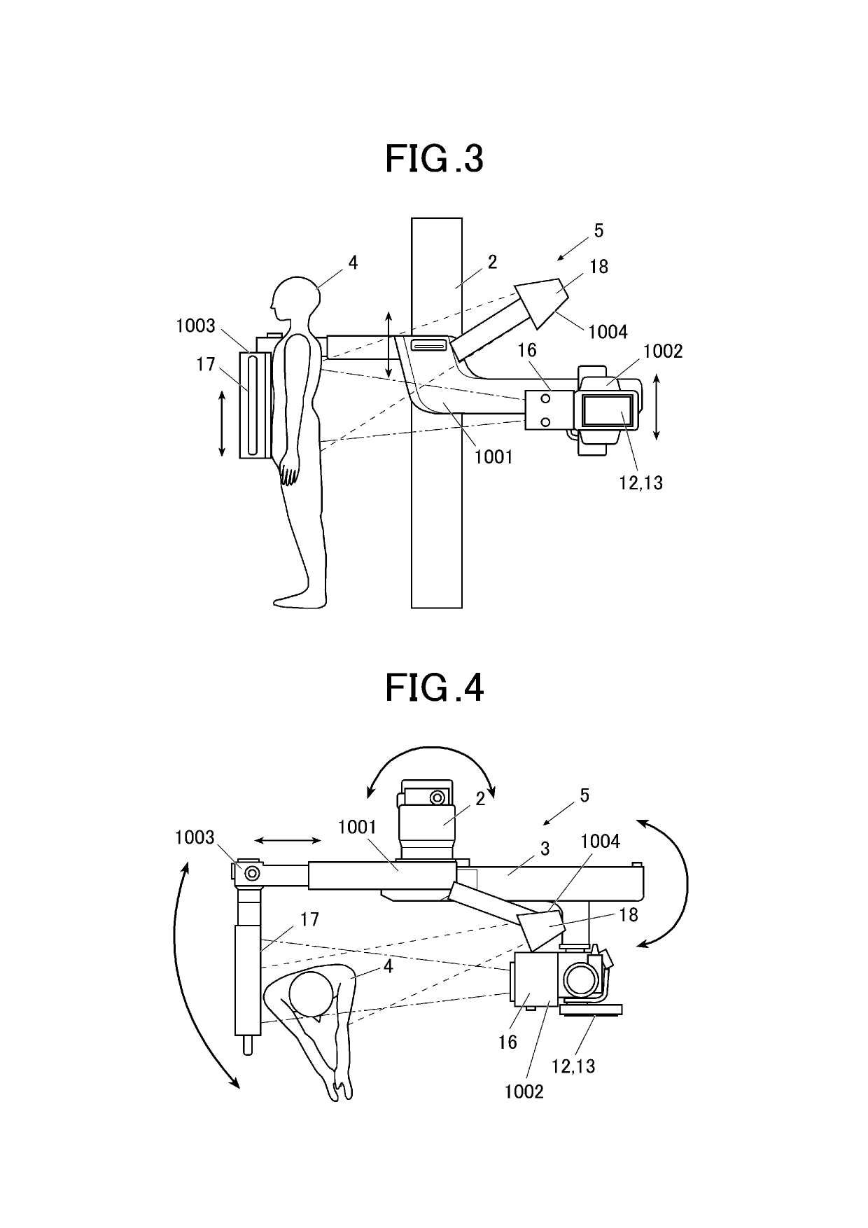

Alternatively, the tube 16 and the optical camera 18 may be separate and movable as separate bodies as in the radiographic imaging apparatus 5 illustrated in FIGS. 3 and 4.

The first movement mechanism 1001, the second movement mechanism 1002, and the third movement mechanism 1003 operate under the control of a controller 11, which will be described later, so that the structure 3, the tube 16, and the FPD 17 are automatically moved with respect to the subject 4. Thus, the tube 16 is automatically positioned with respect to the subject 4.

### <Components of Radiographic Imaging Apparatus 1>

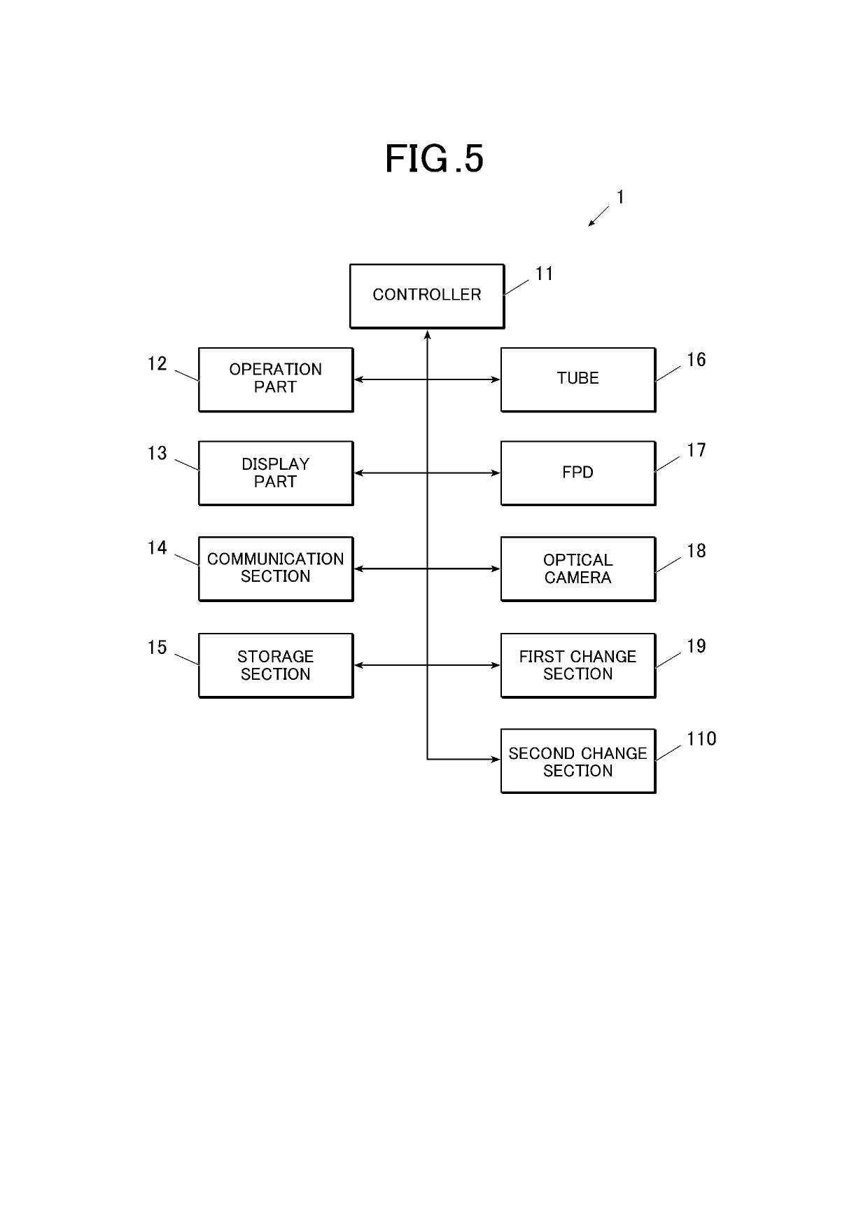

Next, each component of the radiographic imaging apparatus 1 according to the present embodiment will be described with reference to FIG. 5. FIG. 5 is a block diagram illustrating a configuration of the radiographic imaging apparatus 1.

As illustrated in FIG. 5, the radiographic imaging apparatus 1 includes the controller 11 (hardware processor), an operation part 12, a display part 13, a communication section 14, a storage section 15, the tube 16, the FPD 17, the optical camera 18, a first change section 19, and a second change section 110.

The controller 11 includes a central processing unit (CPU and a random-access memory (RAM). The CPU of the controller 11 reads a system program and various processing programs stored in the storage section 15 in response to an input from the operation part 12 and loads the programs into the RAM. The CPU of the controller 11 executes various processes in accordance with the loaded program.

The controller 11 functions as a determination section that determines, based on multiple optical images, whether the relative position between the tube and the subject is good. The multiple optical images include not only optical images captured as still images but also frame images captured as a moving image.

The multiple optical images are captured at different imaging positions and imaging angles. The optical images also include optical images captured at different angles of view, for example.

As the determination based on the multiple optical images, the determination may be done multiple times based on one optical image, or the determination may be done multiple times, based on multiple optical images.

The controller 11 functions as a position determination section that determines the relative positions between the tube and the subject, based on the determination result by the determination section.

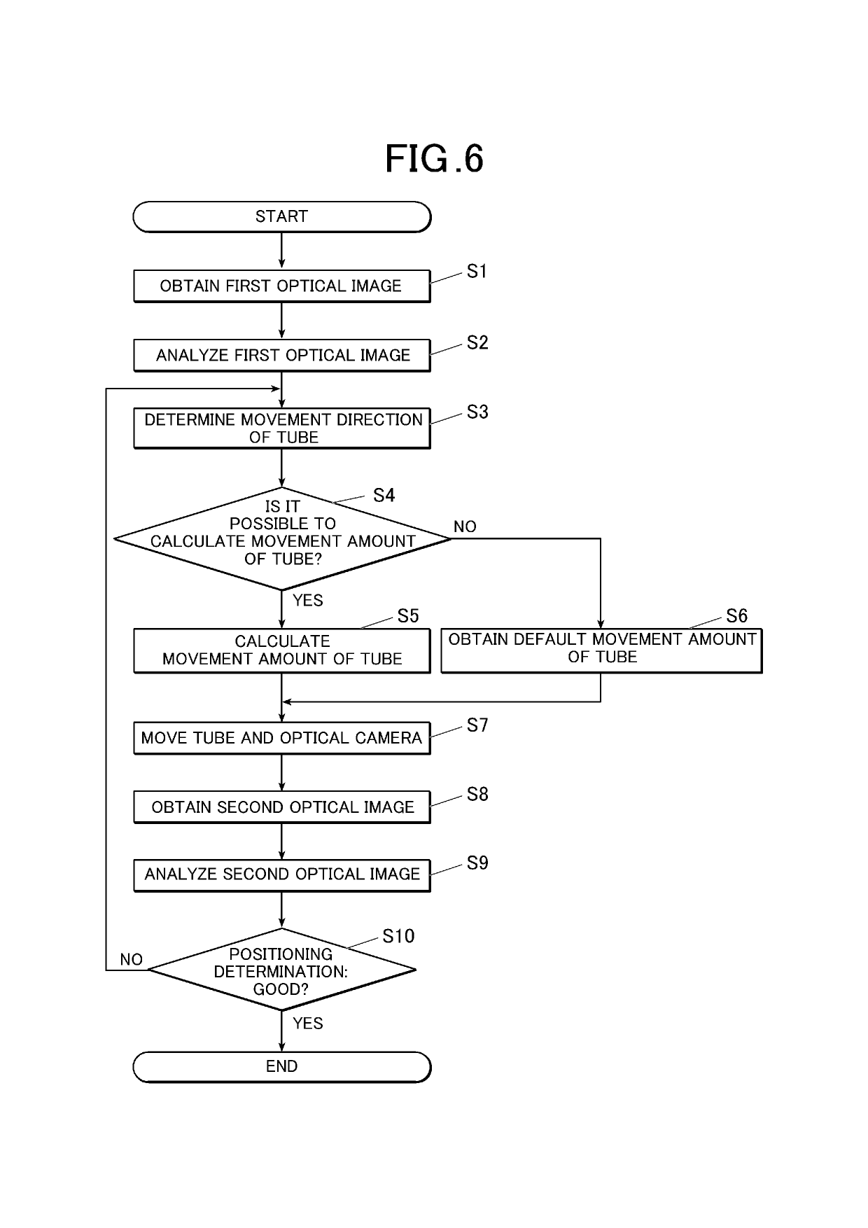

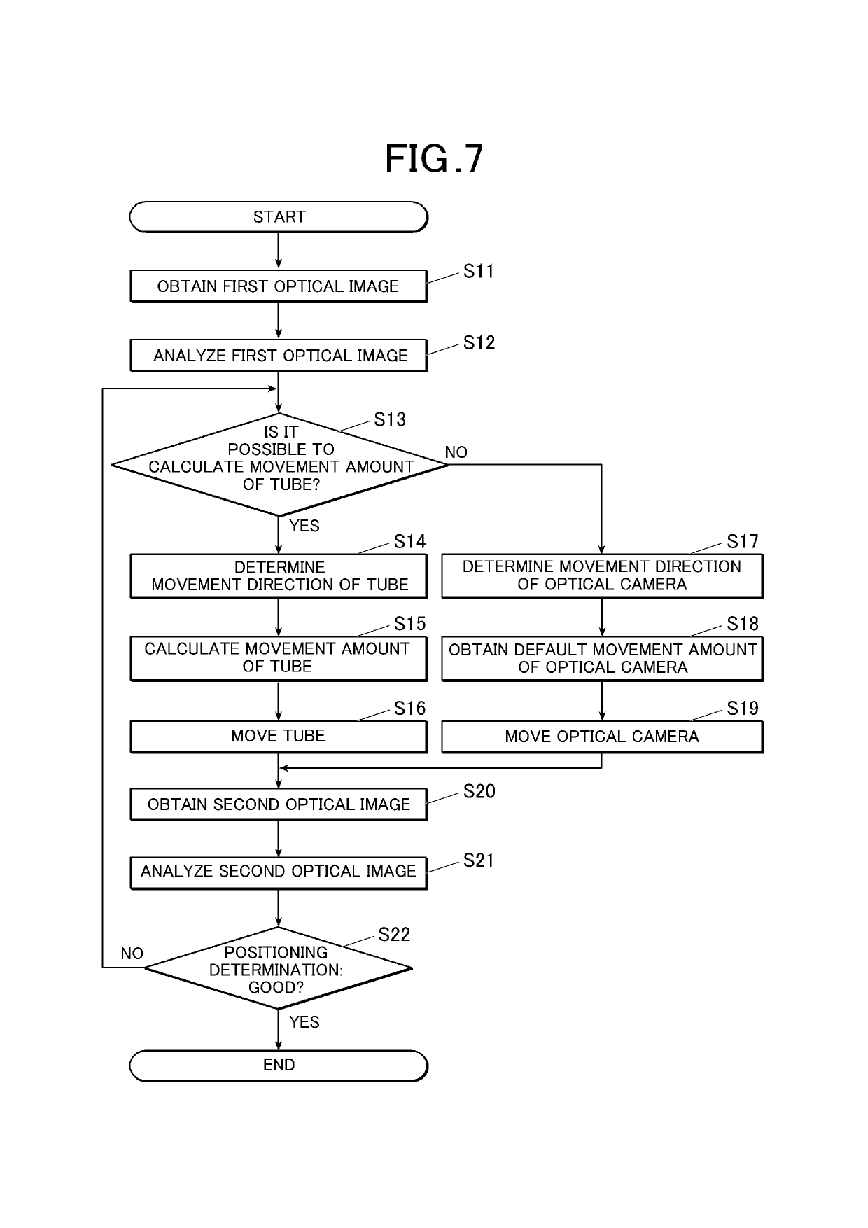

To determine the relative positions, the optical camera and the tube may be moved first, and then their positions may be determined to positions where the positioning is judged to be satisfactory (the positioning determination process in FIG. 6); or the tube may be moved to the position where the positioning is judged to be satisfactory (the positioning determination process in FIG. 7).

The position determination process is performed by using various analysis models, such as a machine learning model.

The analysis model has been learned in advance using optical images of the subject 4 captured by the optical camera 18 as input information and relative positional information of the tube 16 and the subject 4 as output information. The relative positional information of the tube 16 and the subject 4 is, specifically, a deviation direction and/or a deviation amount from their correct positions. At least the deviation direction is output.

The positional information of the tube 16 and the optical camera 18 can be obtained from operation information of the first movement mechanism 1001, the second movement mechanism 1002, and the fourth movement mechanism 1004 that move the tube 16 and the camera 18.

The number of optical images input to the analysis model is not limited to one. Multiple optical images may be input to the analysis model.

As the analysis method, the controller 11 may estimate the position and posture of a person from optical images (Japanese Unexamined Patent Publication No. 2018-206321), for example.

The operation part 12 includes a touch screen or the like in which transparent electrodes are arranged in a lattice shape so as to cover the surface of the display part 13. The operation part 12 is operable by an operator. The operation part 12 outputs various signals based on an operation performed by the operator to the controller 11.

The display part 13 includes a monitor, such as a liquid crystal display (LCD) and a cathode ray tube (CRT). The display part 13 displays contents in accordance with an instruction of a display signal input by the controller 11.

The communication section 14 performs data transmission and reception to and from various devices including the FPD 17 over wired or wireless communication. Examples of the wireless communication method include a wireless local area network (LAN), Bluetooth, and infrared rays communication. The communication section 14 may use other wireless communication methods or may be able to support multiple wireless communication methods.

The various apparatuses include a radiology information system (RIS), an electronic medical record system, and a picture archiving and communication system (PACS) in addition to the FPD 17.

The storage section 15 includes a nonvolatile semiconductor memory and/or a hard disk. The storage section 15 stores various programs executed by the controller 11, parameters necessary for processing by the programs, or data such as processing results.

The tube 16 (radiation source) is driven by a drive section (not illustrated). The drive section is a circuit that drives the tube 16. The drive section and the tube 16 are connected via a cable.

The tube 16 irradiates the subject 4 with radiation. The radiation is, for example, X-rays.

The FPD 17 is disposed opposite the tube 16 with the subject 4 in-between. The FPD 17 generates charges corresponding to the radiation that has been emitted by the tube 16 and transmitted through the subject 4 and reads out the generated charges as image data.

The optical camera 18 performs imaging of the subject 4 to obtain an optical image. The optical camera 18 can obtain not only still images but also moving images consisting of optical images by continuously imaging the subject 4.

The first change section 19 includes the first movement mechanism 1001 and/or the second movement mechanism 1002. The first change section 19 moves the tube 16 to change the relative position of the tube 16 with respect to the subject 4.

The first change section 19 can obtain the positional information of the tube 16 from the operation information of the first movement mechanism 1001 and/or the second movement mechanism 1002.

The second change section 110 includes the first movement mechanism 1001 and/or the fourth movement mechanism 1004. The second change section 110 moves the optical camera 18 to change the relative position of the optical camera 18 with respect to the subject 4.

The second change section 110 can obtain the positional information of the optical camera 18 from the operation information of the first movement mechanism 1001 and/or the fourth movement mechanism 1004.

When the tube 16 and the optical camera 18 are integrally formed, the first change section 19 also functions as the second change section 110.

### <Positioning Determination Process: Case 1>

Next, the positioning determination process in the present embodiment will be described with reference to FIG. 6.

In the positioning determination process, the controller 11 determines whether the position of the tube 16 with respect to the subject 4 is correct, and the tube 16 is automatically positioned with respect to the subject 4.

The positioning determination process illustrated in FIG. 6 is for the radiographic imaging apparatus 1 that includes the tube 16 and the optical camera 18 as one integrated unit.

The default movement amount of a tube 16, which is described later, has been input by the user via the operation part 12 and stored in the storage section 15 beforehand.

First, the controller 11 controls the optical camera 18 to obtain a first optical image of the subject 4 from the optical camera 18 (step S1). The first optical image may be a single image or multiple images. To obtain multiple first optical images, the controller 11 controls the movement mechanisms to change the position of the optical camera 18, and obtains multiple first optical images.

Next, the controller 11 analyzes the first optical image(s) by using the analysis model (step S2). The controller 11 obtains the deviation direction and/or the deviation amount. The controller 11 obtains at least the deviation direction.

The deviation direction includes an up-down direction, front-back direction, left-right direction, inclination (rotation direction) with respect to the subject 4, and a combination of these directions.

Next, the controller 11 determines the movement direction of the tube 16, based on the obtained deviation direction (step S3).

Next, the controller 11 determines whether it is possible to calculate the movement amount of the tube 16, based on the obtainment state of the deviation amount (step S4). If the movement amount can be calculated (step S4: YES), the controller 11 proceeds to step S5. If the movement amount cannot be calculated (step S4: NO), the controller 11 proceeds to step S6.

Next, the controller 11 calculates the movement amount of the tube 16, based on the obtained deviation amount (step S5). If there is a difference between the movement amount obtained by the analysis and the actual movement amount in the apparatus, the controller 11 may reflect the difference to the deviation amount in calculating the movement amount.

Next, the controller 11 obtains the default movement amount of the tube 16 from the storage section 15 (step S6).

Next, the controller 11 moves the tube 16 and the optical camera 18, which are formed as one integrated unit, by using the first change section 19 (step S7).

First, the controller 11 controls the optical camera 18 to obtain a second optical image of the subject 4 from the optical camera 18 (step S8). The second optical image may be a single image or multiple images. To obtain multiple second optical images, the controller 11 controls the movement mechanisms to change the position of the optical camera 18 and obtains multiple second optical images.

Next, the controller 11 analyzes the second optical image by using the analysis model (step S9). The controller 11 obtains the deviation direction and/or the deviation amount. The controller 11 obtains at least the deviation direction.

Next, the controller 11 determines whether the positioning of the tube 16 with respect to the subject 4 is good (step S10). When the deviation amount is less than a threshold value (Step S10: YES), the controller 11 determines that the positioning is good and ends the positioning determination process. When the deviation amount is greater than or equal to the threshold value or when the deviation amount is not obtained (step S11: NO), the controller 11 determines that the positioning is not good and proceeds to step S3.

Thus, the tube 16 and the optical camera 18, which are formed as one integral unit, are gradually moved up and down, back and forth, right and left, and in the rotation direction, and the positioning determination is repeated. Accordingly, the relative positions between the tube 16 and the subject 4 can be automatically and optimally determined.

The inclination with respect to the subject 4 cannot be calculated from one optical image. According to the above way, the deviation direction including the inclination and the deviation amount including the inclination amount can be calculated.

### Positioning Determination Process: Case 2

Next, the positioning determination process in the present embodiment will be described with reference to FIG. 7.

The positioning determination process illustrated in FIG. 7 is for the radiographic imaging apparatus 1 that includes the tube 16 and the optical camera 18 as separate bodies.

The default movement amount of the optical camera 18, which is described later, has been input by the user via the operation part 12 and stored in the storage section 15 beforehand.

Since steps S11 and S12 are the same as steps S1 and S2 of FIG. 6, description thereof will be omitted.

Since steps S20 to S22 are the same as steps S8 to S10 of FIG. 6, description thereof will be omitted.

The controller 11 determines whether it is possible to calculate the movement amount of the tube 16, based on the obtainment state of the deviation amount in step S12 (step S13). When the movement amount can be calculated (step S13: YES), the controller 11 proceeds to step S14. When the movement amount cannot be calculated (step S13: NO), the controller 11 proceeds to step S17.

Following is the description of a case where the movement amount of the tube 16 can be calculated.

First, the controller 11 determines the movement direction of the tube 16, based on the obtained deviation direction (step S14).

Next, the controller 11 calculates the movement amount of the tube 16, based on the obtained deviation amount (step S15). If there is a difference between the movement amount obtained by the analysis and the actual movement amount in the apparatus, the controller 11 may reflect the difference to the deviation amount in calculating the movement amount.

Next, the controller 11 moves the tube 16 by using the first change section 19 (step S16). The controller 11 proceeds to step S20.

Following is the description of a case where the movement amount of the tube 16 cannot be calculated.

First, the controller 11 determines the movement direction of the optical camera 18, based on the deviation direction of the tube 16 obtained in step S12 (step S17). That is, the controller 11 determines the movement direction of the optical camera 18 in which the movement amount of the tube 16 is estimated to be calculable.

Next, the controller 11 obtains the default movement amount of the optical camera 18 from the storage section 15 (step S18).

Next, the controller 11 moves the optical camera 18 by using the second change section 110 (step S19). The controller 11 proceeds to step S20.

Thus, the optical camera 18 is gradually moved up and down, back and forth, right and left, and in the rotation direction; the positioning determination is repeated; and the tube 16 is moved to the position where the positioning is determined to be good. Accordingly, the relative positioning of the tube 16 with respect to the subject 4 can be automatically optimized.

### <Others>

In the positioning determination process, the position where positioning is determined to be good for the first time in the positioning determination (step S10 and step S22) is determined to be a final position. However, the present disclosure is not limited to this example.

For example, the controller 11 may control the movement mechanisms to change the initial position of the tube 16 and/or the optical camera 18, execute the positioning determination process multiple times, and determine the position where the determination result is the best to be the final position. At the position where the determination result is the best, the deviation amount is the smallest.

In the above description, the optical image obtained by the optical camera 18 is used. However, the present disclosure is not limited thereto.

For example, a radiographic image obtained by low-dose exposure with the tube 16 may be used. For another example, in a case of re-imaging, if the subject 4 has not moved after the last imaging until the next re-imaging, the radiographic image at the time of the last imaging may be used. For another example, whether the positioning is good may be determined, based on serial signals without using a radiographic image (frame image).

Furthermore, although the tube 16 and/or the optical camera 18 are automatically moved under the control of the controller 11 in the above description, the present disclosure is not limited to this.

A semi-automatic configuration may be adopted, wherein: a mechanism that fixes the movement direction of the tube 16 and/or the optical camera 18 is provided; the movement direction of the tube 16 and/or the optical camera 18 is fixed by the controller 11; and the tube 16 and/or the optical camera 18 is moved manually by the user. The controller 11 may indicate the movement amount to the user by displaying the movement amount on the display part 13.

Furthermore, the controller 11 (determination section) may determine imaging conditions by using an analysis model (e.g., a machine learning model) that learns order information and sensor information (information obtained by the optical camera 18 and/or information on the position of the tube obtained by the first change section) as inputs and imaging conditions as outputs, such as radiation emission conditions by the tube 16 and radiation accumulation and reading conditions by the FPD 17. Thus, optimal imaging conditions are derived.

The imaging conditions include, for example, an irradiation field size, the position of the tube center, the incident angle, and X-ray irradiation conditions (e.g., mAs value, tube voltage, filter type).

For example, the controller 11 estimates the thickness of the subject, based on body part information and an optical image(s), and determines the tube voltage, tube current, and irradiation time.

For example, the controller 11 estimates the contour of the region of interest, based on the body part information and the optical image, and determines the irradiation field size.

For example, the controller 11 can accurately determine the position of the tube by finely correcting the position of the tube center and the incident angle, based on the deviation direction and/or the deviation amount of the tube and the subject obtained from the optical image and the positional information of the tube.

Further, instead of the optical camera 18, a sensor such as a depth camera or a lidar scanner that can detect a relative positional deviation amount between the tube and the subject may be used.

### <Effect>

As described above, the radiographic imaging apparatus 1 includes: the optical camera 18 that captures optical images; the first change means (first change section 19) that changes the relative position of the tube with respect to the subject; and the determination section (controller 11) that determines, based on multiple optical images, whether the relative positioning of the tube with respect to the subject is good. Based on the determination result by the determination section, the radiographic imaging apparatus 1 determines the relative position of the tube with respect to the subject.

Thus, the relative positions between the tube and the subject can be automatically and optimally determined.

The radiographic imaging method is performed by the radiographic imaging apparatus 1 that includes: the optical camera 18 that captures optical images; and the first change means (first change section 19) that changes the relative position of the tube with respect to the subject. The method includes: a positioning determination step (Steps S10 and S22) to determine whether the relative positioning of the tube with respect to the subject is good, based on multiple optical images; and a position determination step (Steps S3 to S6, S13 to S15) to determine the relative position of the tube with respect to the subject, based on the determination result in the positioning determination step.

Thus, the relative positions between the tube and the subject can be automatically and optimally determined.

The program is for the radiographic imaging apparatus 1 that includes: the optical camera 18 that captures optical images; and the first change means (first change section 19) that changes the relative position of the tube with respect to the subject. The program causes a computer (controller 11) of the radiographic imaging apparatus 1 to determine whether the relative positioning of the tube with respect to the subject is good, based on multiple optical images and to determine the relative position of the tube with respect to the subject, based on the determination result by the computer.

Thus, the relative positions between the tube and the subject can be automatically and optimally determined.

Although the present disclosure has been described in detail based on the embodiment, the present disclosure is not limited to the above-described embodiment. The embodiment can be modified without departing from the spirit and scope of the invention.

Although an example in which a semiconductor nonvolatile memory, a hard disk, or the like is used as a computer-readable medium of the program according to the present disclosure has been disclosed in the above description, the present disclosure is not limited to this example.

As other computer-readable media, a nonvolatile memory, such as a flash memory, and a portable recording medium, such as a CD-ROM, are also applicable.

As a medium for providing data of the program according to the present disclosure via a communication line, a carrier wave is also applied to the present disclosure.

Although embodiments of the present disclosure have been described and illustrated in detail, the disclosed embodiments are made for purposes of illustration and example only and not limitation. The scope of the present disclosure should be interpreted by terms of the appended claims.

1. A radiographic imaging apparatus comprising:
an optical camera that obtains optical images;
a first change section that changes a relative position of a tube with respect to a subject; and
a hardware processor,
wherein the hardware processor determines whether relative positioning of the tube with respect to the subject is good, based on the optical images, and determines the relative position of the tube with respect to the subject, based on the determination on the positioning.

2. The radiographic imaging apparatus according to claim 1, wherein the first change section is a tube movement section that moves the tube, based on the optical images, to change the relative position of the tube with respect to the subject.

3. The radiographic imaging apparatus according to claim 1, wherein:
the hardware processor determines whether the positioning is good, based on a first optical image captured by the optical camera at a first position,
the first change section changes the relative position of the tube with respect to the subject, based on the determination with the first optical image, and
the hardware processor again determines whether the positioning is good, based on a second optical image captured by the optical camera at a second position after the relative position of the tube with respect to the subject is changed.

4. The radiographic imaging apparatus according to claim 1, wherein in determining whether the positioning is good, the hardware processor determines whether an imaging position is good and/or whether an imaging angle is good.

5. The radiographic imaging apparatus according to claim 1, further comprising a second change section that changes a relative position of the optical camera with respect to the subject.

6. The radiographic imaging apparatus according to claim 4, wherein the first change section and a second change section are one change section.

7. The radiographic imaging apparatus according to claim 1, wherein the hardware processor determines an imaging condition of radiographic imaging by analyzing order information and sensor information as inputs.

8. The radiographic imaging apparatus according to claim 7, wherein the sensor information is obtained by the optical camera and/or the first change section.

9. The radiographic imaging apparatus according to claim 7, wherein the imaging condition includes at least one of an irradiation field size, a position of a center of the tube, an incident angle, and an X-ray irradiation condition.

10. The radiographic imaging apparatus according to claim 7, wherein the analysis is performed by machine learning.

11. The radiographic imaging apparatus according to claim 9, wherein the X-ray irradiation condition includes a mAs value, a tube voltage, and a filter type.

12. A radiographic imaging method for a radiographic imaging apparatus that includes an optical camera that obtains optical images and a first change section that changes a relative position of a tube with respect to a subject, the method comprising:
determining whether relative positioning of the tube with respect to the subject is good, based on the optical images; and
determining the relative position of the tube with respect to the subject, based on the determination on the positioning.

13. A non-transitory computer-readable storage medium storing a program for a radiographic imaging apparatus that includes an optical camera that obtains optical images and a first change section that changes a relative position of the tube with respect to a subject, the program causing a computer of the radiographic imaging apparatus to:
determine whether relative positioning of the tube with respect to the subject is good, based on the optical images and
determine the relative position of the tube with respect to the subject, based on the determination on the positioning.
